(19) United States
(12) Patent Application Publication
(10) Pub. No.: US 2014/0371852 A1
(43) Pub. Date: Dec. 18, 2014

(54) SCLERAL FIXATION BAG

(71) Applicants: Eli Aharoni, Tel Aviv (IL); Mark Packer, Charlotte, VT (US)

(72) Inventors: Eli Aharoni, Tel Aviv (IL); Mark Packer, Charlotte, VT (US)

(21) Appl. No.: 13/918,970

(22) Filed: Jun. 16, 2013

(51) Int. Cl. A61F 2/16

(52) U.S. Cl. CPC A61F 2/1613
USPC 623/6.51; 623/6.43

(57) ABSTRACT

A method is described for introducing a scleral fixation bag into an eye. The scleral fixation bag includes haptics that extend therefrom. The method includes inserting and affixing the haptics into sclerotomies formed in the eye; the haptics affix the scleral fixation bag without a need for natural capsular bag support. The haptics of the scleral fixation bag include a slender loop element and a distal hook portion that extends from a distal end of the slender loop element. The distal hook portion has an anterior edge tilted towards the central anterior-posterior axis of the scleral fixation bag.

### FIELD OF THE INVENTION

The present invention relates generally to intraocular devices, and particularly to a scleral fixation bag, which can be used, inter alia, to mount therein intraocular lenses (IOLs) when no intact capsular bag is present.

### BACKGROUND OF THE INVENTION

In a typical cataract surgery, the natural lens is removed by phacoemulsification from the eye. The lens material is broken up and vacuumed out of the eye, but the capsular bag is left generally intact. The remaining capsular bag is very important because it houses and supports an IOL, which can be acted on by the zonules and ciliary muscles for possible accommodation.

However, the capsular bag does not always provide sufficient support for the IOL. When performing cataract surgery for eyes with severe zonular dehiscence or lens subluxation, surgeons need to employ a surgical technique to fixate an intraocular lens (IOL) without adequate capsular support. Several methods have been employed by surgeons to address the problem of insufficient capsular support. One method is implantation of an open-loop anterior chamber IOL. Another method involves iris suture fixation or scleral suture fixation of a posterior chamber IOL. Yet another method involves scleral fixation of a capsular tension ring with the IOL.

However, all of these methods are applicable when the capsular bag is still intact. There are situations when no capsular bag is present. For example, a lens exchange is required to replace an IOL with another IOL. Removing the first IOL can severely damage the capsular bag to the point where a remnant bag or no bag is present for holding the replacement IOL.

### SUMMARY OF THE INVENTION

The present invention seeks to provide improved methods and devices for mounting IOLs or other intraocular devices even if no capsular bag is present. As is described further in detail hereinbelow, the invention provides a scleral fixation bag which serves as a mounting platform for intraocular devices, even if no natural bag is present.

There is thus provided in accordance with an embodiment of the present invention a method including introducing a scleral fixation bag into an eye, the scleral fixation bag including haptics that extend therefrom, and inserting and affixing the haptics into sclerotomies formed in the eye, wherein the haptics affix the scleral fixation bag without a need for capsular bag support. The method is useful even if no capsular bag is present. The method can further include mounting an intraocular device into a channel formed in the scleral fixation bag.

Non-limiting features of the invention include the following, among others:

A scleral fixation bag includes haptics that extend therefrom, each of the haptics including a slender loop element with a proximal end attached to the scleral fixation bag, the slender loop element having a length, width and thickness, the length following a curved path and the width being generally parallel to a central anterior-posterior axis of the scleral fixation bag. A distal hook portion extends from a distal end of the slender loop element, the distal hook portion including an anterior edge tilted towards the central anterior-posterior axis of the scleral fixation bag.

The distal hook portion includes a posterior edge tilted away from the central anterior-posterior axis of the scleral fixation bag.

The distal hook portion is round at its distal tip and tapers to be thinner from where it extends from the slender loop element.

The distal hook portion is thinner than the slender loop element.

The thickness of the slender loop elements is less than the width.

The distal hook portion is formed with a through hole.

The proximal end of the slender loop element is generally tangential to an outer periphery of the scleral fixation bag.

The distal hook portion and the slender loop element have different rigidity.

The slender loop element and the scleral fixation bag have different rigidity.

The anterior-most surface of the distal hook portion is not posterior to an anterior-most surface of the intraocular device.

The anterior-most surface of the slender loop element is not posterior to an anterior-most surface of the scleral fixation bag.

The scleral fixation bag includes a channel for receiving therein an IOL.

### BRIEF DESCRIPTION OF THE DRAWINGS

The present invention will be understood and appreciated more fully from the following detailed description taken in conjunction with the appended drawings in which:

FIG. 1 is a simplified pictorial illustration of a scleral fixation bag with haptics, constructed and operative in accordance with an embodiment of the present invention;

FIG. 2 is a simplified illustration of the scleral fixation bag of FIG. 1 implanted in an eye, viewed transverse to the anterior-posterior axis, wherein the haptics are fixed in a curved sclerotomy, in accordance with an embodiment of the present invention;

FIG. 3 is a simplified side-view illustration of a portion of the scleral fixation bag;

FIG. 4 is a simplified side-view illustration of an intraocular device (IMT) mounted in the scleral fixation bag, in accordance with an embodiment of the present invention;

FIG. 5 is a simplified pictorial illustration of a scleral fixation bag with haptics, constructed and operative in accordance with another embodiment of the present invention;

FIG. 6 is a simplified pictorial illustration of an IOL mounted in the scleral fixation bag that cooperates with another IOL mounted in the posterior chamber, e.g., in the ciliary muscles, in accordance with another embodiment of the present invention; and

FIG. 6A is an enlarged view of a portion of the fixation bag, showing a posterior platform attached to the scleral fixation bag.

### DETAILED DESCRIPTION OF THE PRESENT INVENTION

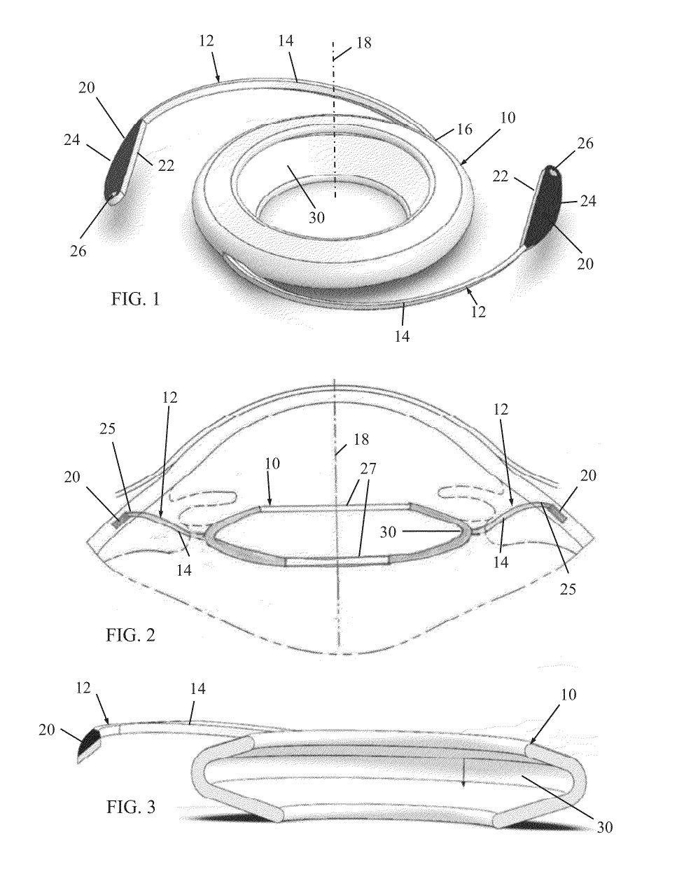

Reference is now made to FIGS. 1-3, which illustrate a scleral fixation bag 10 with haptics 12, constructed and operative in accordance with an embodiment of the present invention. Scleral fixation bag 10 is shown as a ring, but it may have other shapes as well.

Each haptic 12 includes a slender loop element 14 with a proximal end 16 attached to scleral fixation bag 10. Slender loop element 14 has a length, width and thickness; its length follows a curved path and its width is generally parallel to a central anterior-posterior axis 18 of scleral fixation bag 10. The outer diameter of scleral fixation bag 10 with haptics 12 is, without limitation, 15 mm for proper mounting in the sclera.

A distal hook portion 20 extends from a distal end of slender loop element 14. Distal hook portion 20 includes an anterior edge 22 tilted towards the central anterior-posterior axis 18 of scleral fixation bag 10. Preferably, but not necessarily, the posterior edge 28 of distal hook portion 24 is tilted away from central anterior-posterior axis 22. Preferably, but not necessarily, distal hook portion 24 is flat. The shape of distal hook portion 24 may match the shape of its insertion place in the sclera. Distal hook portion 24 is rounded at its distal tip and tapers to be thinner from where it extends from slender loop element 18. Distal hook portion 24 may be formed with a through hole 30, for grasping or dialing or other adjustments. Through hole 30 is preferably central and not shifted to one side. In one embodiment, distal hook portion 20 is thinner than slender loop element 14. In one embodiment, the thickness of slender loop elements 14 is less than its width; in other embodiments they are equal. The distal hook portion may be connected to a suture (not shown) in different surgical procedures.

The proximal end of slender loop element 14 is generally tangential to the outer periphery of scleral fixation bag 10.

The scleral fixation bag 10 and haptics 12 may be made from the same or different materials, and each may be made of one or more materials. The materials used are biocompatible and optically transparent, and can be hydrophilic or hydrophobic. The material may be rigid or flexible, hard or soft, such as without limitation, methacrylates (e.g., polymethyl methacrylate), olefins (e.g., polypropylene), and silicones. For example, without limitation, scleral fixation bag 10 may be made of silicone whereas haptics 12 may be made of PMMA.

The distal hook portion 20 and the slender loop element 14 can have different rigidity. Similarly, slender loop element 14 and scleral fixation bag 10 can have different rigidity. Fixation bag 10 can be made of a flexible folding material for easy insertion during the surgical procedure.

It is noted that the anterior-most surface of distal hook portion 20 is not posterior to the anterior-most surface of scleral fixation bag 10 (that is, it is either flush with it or more anterior). Similarly, the anterior-most surface of the slender loop element 14 is not posterior to the anterior-most surface of scleral fixation bag 10.

Scleral fixation bag 10 includes an open central aperture 27 (FIG. 2).

In accordance with an embodiment of the present invention the scleral fixation bag 10 includes a channel 30 for receiving therein an intraocular device, such as an intraocular lens (IOL) or implantable telescope, not shown. Channel 30 may extend completely around the inner periphery of scleral fixation bag 10 or may extend over one or more discrete angular segments of the inner periphery of scleral fixation bag 10.

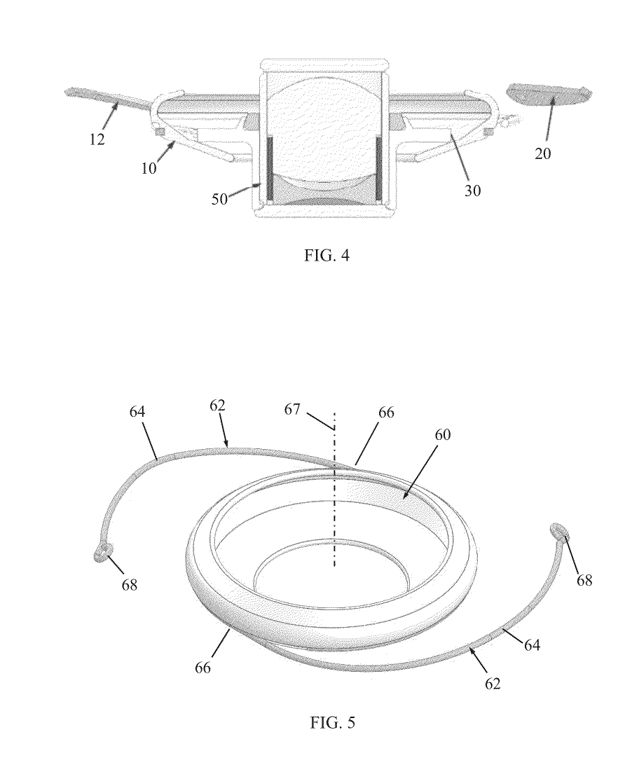

The scleral fixation bag 10 can be introduced through an incision in the limbus, for example. The haptics 12 are fixed in curved sclerotomies 25 and properly center the fixation bag 10. An IOL (such as, but not limited to, a monofocal IOL, multifocal IOL, accommodating IOL and others) or other intraocular device (such as an implantable miniature telescope (IMT) 50, seen in FIG. 4) can be easily mounted in channel 30. The fixation bag 10 is held properly by the haptics 12 in sclerotomies 25, even without any capsular bag being present.

Reference is now made to FIG. 5, which illustrates a scleral fixation bag 60 with haptics 62, constructed and operative in accordance with another embodiment of the present invention. As in the other embodiments of the invention, each haptic 62 includes a slender loop element 64 with a proximal end 66 attached to scleral fixation bag 60 and a distal hook portion 68 whose anterior edge is tilted towards the central anterior-posterior axis 67 of scleral fixation bag 60. In this embodiment, slender loop element 64 is a slender wire and distal hook portion 68 is a rounded eyelet bent or otherwise formed from the end of the slender wire.

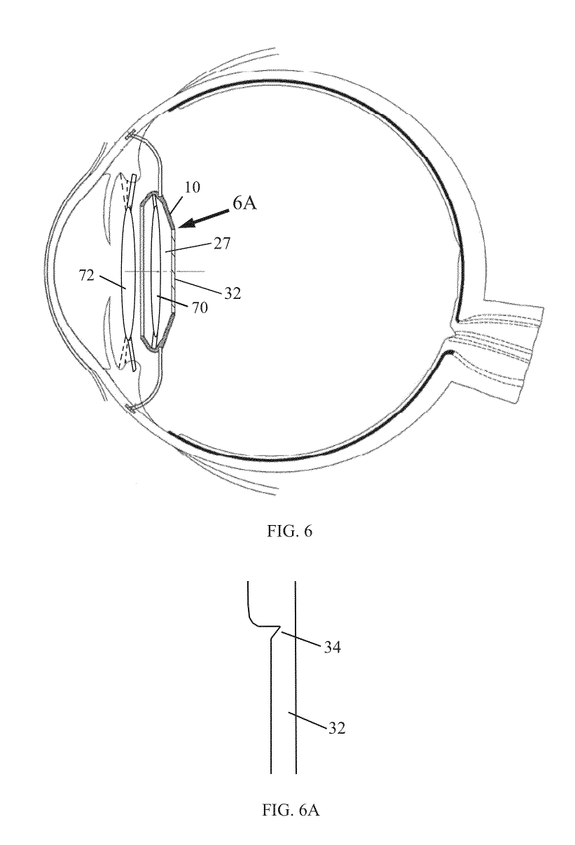

The scleral fixation bag of the present invention can be useful in providing a relatively static platform for mounting therein an IOL, which can be used in conjunction with another IOL mounted posterior to the iris and anterior to the scleral fixation bag, such as in the ciliary muscles, ciliary process or ciliary sulcus. This creates a doublet lens with superior accommodative power than previous lenses. This is because the posterior of the two lenses, that is, the lens mounted in the scleral fixation bag, is held much more statically than the anterior of the two lenses; thus the anterior lens moves much more with respect to the posterior lens than prior art doublet IOLs which are both held in the capsular bag and have much less relative movement because both lenses move. Such an embodiment is illustrated in FIG. 6, wherein an IOL 70 is mounted in scleral fixation bag 10 and another IOL 72 is mounted in the posterior chamber posterior to the iris and anterior to the scleral fixation bag 10, such as in the ciliary muscles, or even in the ciliary sulcus (indicated by broken lines).

FIG. 6 also illustrates another option, namely a posterior platform 32 attached to the scleral fixation bag 10. Platform 32 may be used to close the open central aperture 27 of bag 10. This may help prevent migration of vitreous humor through the fixation bag 10. Platform 32 may be formed with an annular groove 34 (seen in the enlarged view of FIG. 6A), so that the portion of platform 32 which covers aperture 27 may be broken off, if desired for a particular application. Platform 32 may have optical power (positive or negative). Platform 32 and scleral fixation bag 10 may be made from the same or different materials. Platform 32 may also be useful in the doublet lens configuration of FIG. 6, in which the platform helps keep the lens static in the fixation bag.

What is claimed is:

1. A method comprising:
introducing a scleral fixation bag into an eye, said scleral fixation bag comprising haptics that extend therefrom; and
inserting and affixing said haptics into sclerotomies formed in the eye, wherein said haptics affix said scleral fixation bag without a need for natural capsular bag support.

2. The method according to claim 1, wherein no capsular bag is present.

3. The method according to claim 1, further comprising mounting an intraocular device into a channel formed in said scleral fixation bag.

4. An intraocular device comprising:
a scleral fixation bag comprising haptics that extend therefrom, each of said haptics comprising a slender loop element with a proximal end attached to said scleral fixation bag, said slender loop element having a length, width and thickness, the length following a curved path and the width being generally parallel to a central anterior-posterior axis of said scleral fixation bag; and
a distal hook portion extending from a distal end of said slender loop element, said distal hook portion comprising an anterior edge tilted towards the central anterior-posterior axis of said scleral fixation bag.

5. The intraocular device according to claim 4, wherein said distal hook portion comprises a posterior edge tilted away from the central anterior-posterior axis of said scleral fixation bag.

6. The intraocular device according to claim 4, wherein said distal hook portion is rounded at its distal tip and tapers to be thinner from where it extends from said slender loop element.

7. The intraocular device according to claim 4, wherein said distal hook portion is flat.

8. The intraocular device according to claim 4, wherein the thickness of said slender loop elements is less than the width.

9. The intraocular device according to claim 4, wherein said distal hook portion is formed with a central through hole.

10. The intraocular device according to claim 4, wherein said distal hook portion and said slender loop element have different rigidity.

11. The intraocular device according to claim 4, wherein an anterior-most surface of said distal hook portion is not posterior to an anterior-most surface of said intraocular device.

12. The intraocular device according to claim 4, wherein said scleral fixation bag comprises a channel for receiving therein a different intraocular device.

13. The intraocular device according to claim 4, wherein a posterior platform is attached to said scleral fixation bag.

14. The intraocular device according to claim 13, wherein said posterior platform is formed with an annular groove.

15. The intraocular device according to claim 4, wherein said posterior platform has optical power.

16. The method according to claim 4, further comprising mounting another intraocular device in the posterior chamber anterior to the intraocular device mounted in said scleral fixation bag.
